(19) United States
(12) Patent Application Publication
(10) Pub. No.: US 2006/0029654 A1
(43) Pub. Date: Feb. 9, 2006

(54) Analgesic patch for sports injury rehabilitation medicine and method to alleviate pain

(76) Inventor: R. Douglas Cassel, Hummlestown, PA (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 11/196,964

(22) Filed: Aug. 4, 2005

Related U.S. Application Data
(60) Provisional application No. 60/598,694, filed on Aug. 4, 2004.

(51) Int. Cl. A61K 9/70; A61K 31/24

(52) U.S. Cl. 424/449; 514/536

(57) ABSTRACT

The present invention relates to a method for topical treatment of pain resulting from a sports injury, including injuries to bones and soft tissue, such as ligaments, muscles or tendons. The method includes the steps of: applying to skin of a subject suffering from pain resulting from a sports injury a transdermal drug delivery system comprising a formulation comprising a pain-relieving amount of a local anesthetic and a pharmaceutically acceptable carrier, wherein the transdermal drug delivery system is applied to a skin site proximate to or in the region of a sports injury, and relieving the pain. The concentrated local anesthetic in the patch penetrates deeply below the skin to the injury site to inhibit pain receptors throughout the damaged body tissue.

### CROSS REFERENCE TO RELATED APPLICATIONS

This application claims the benefit of the filing date of provisional application No. 60/598,694, filed Aug. 4, 2004, which is incorporated herein by reference.

### FIELD OF THE INVENTION

The present invention relates to a topical patch for alleviating pain associated with sports-related physical activity.

### BACKGROUND OF THE INVENTION

Sports injuries, which are injuries that result from acute trauma or repetitive stress associated with athletic activities, can affect bones or soft tissue, such as ligaments, muscles, or tendons. Adults are less likely to suffer sports injuries than are children, whose vulnerability is heightened by immature reflexes, an inability to recognize and evaluate risks, and their underdeveloped coordination. Although injury rates are highest for athletes who participate in contact sports, the most serious sports injuries are associated with individual activities. One-half to two-thirds of childhood sports injuries occur during practice or in the course of unorganized athletic activity. See Taylor, Robert B., Ed. Family Medicine Principles and Practice, New York: Springer-Verlag (1994).

About 95% of sports injuries are minor soft tissue traumas. Id. The most common sports injury is a bruise (contusion), which is caused when blood collects at the site of an injury and discolors the skin. Id. Sprains, which account for one-third of all sports injuries, are an injury to a ligament; a ligament is a band or sheath of fibrous tissue connecting two or more bones, cartilages or other structures or serving as support for fasciae or muscles. Stedman's Medical Dictionary, 27^{th} Ed. (Lippincott, Williams & Wilkins: New York) (2000). A strain is a partial or complete tear of muscle (a tissue composed of contractile cells that enables the various body parts to move) or tendon (a strong connective tissue that links muscle to bone). Inflammation of a tendon (“tendonitis”) and inflammation of one of the fluid-filled sacs that allow tendons to move easily over bones (“bursitis”) usually result from minor stresses that repeatedly aggravate the same part of the body. These conditions often occur at the same time.

Common causes of sports injuries include: athletic equipment that malfunctions or is used incorrectly; falls, forceful high-speed collisions between players; and wear and tear on areas of the body that are continually subjected to stress. Taylor, Family Med. Principles & Practice (1994). Symptoms of sports injury include: instability or obvious dislocation of a joint pin, swelling and weakness. Symptoms that persist, intensify or reduce the athlete's ability to play without pain should be evaluated by a sports medicine physician. Prompt diagnosis often can prevent minor injuries from becoming major problems or causing long-term or lasting damage.

Treatment for minor soft tissue injuries generally consists of compressing the injured area with an elastic bandage; elevation; applying ice; and rest. Anti-inflammatory medications and exercises to correct muscle imbalances usually are used to treat tendonitis. If the athlete keeps stressing inflamed tendons, they may rupture; casting or surgery sometimes is necessary to correct this condition. Orthopedic surgery may be required to repair serious sprains and strains.

Athletes who have been injured are usually advised to limit their activities until their injuries are healed. The physician may suggest special exercises or behavior modifications for athletes who have had several injuries. Athletes who have been severely injured may be advised to stop playing altogether.

The goals of treatment for overuse injuries are to control inflammation and to restore normal use and mobility. Athletic injuries are customarily treated through use of orally applied analgesics combined with physical therapy. The oral analgesic is carried into the patient's circulatory system and prevents the recognition of pain systemically by interrupting the transmission of pain signals from sensory neurons to the pain centers in the brain. Traditional oral analgesics include opioids (narcotics) such as morphine, codeine, methadone, demerol or darvon; and non-steroidal anti-inflammatory drugs (NSAIDs), such as aspirin, ibuprofen or naproxen.

The systemic use of these drugs carries patient risk. Opioid use causes a variety of undesired side-effects, including sedation, dizziness, depression, nausea and constipation. Prolonged opioid usage carries a risk of patient addiction. The large and sometimes prolonged doses of non-steroidal anti-inflammatory drugs (“NSAIDs”) required to treat intense pain from athletic injuries can cause gastric disorders, erosion of the stomach lining and intestinal mucus membrane, nephrotoxicity, hepatotoxicity, as well as internal bleeding. Orally administered drugs also cause side-affects that restrict physical activity (primarily in the case of opioids, due to sedation) and inhibit effective physical therapy. For an athlete, pain management drugs can inhibit athletic performance or be grounds for disqualification from athletic competition.

Pain from athletic injuries also can be treated locally by delivering a pain reliever directly to the injury site through use of a local anesthetic. Local anesthetics block the transmission of pain receptor signals in the immediate area of the injury.

Direct application of local anesthetics to an injury site is difficult. Effective dosages can be applied by injection into the injury site. Use of this method is not preferred, as injections into an injury site are painful, may aggravate the injury and require professional administration. There is also the drawback that a local anesthetic injected into a highly vascularized area of the body can be carried away by the circulatory system and create the same risks as systemically administered anesthetics. This risk is increased when local anesthetic dosages are increased to manage intense pain.

The topical administration of a local anesthetic overcomes some of the drawbacks of injection. There is no need for the painfully invasive procedure and professional administration is not needed. The risk of the locally applied anesthetic acting as a systemically administered drug also is much reduced.

Dermal patches are well known to administer local anesthetics topically to patients at wound sites and to treat skin ailments. Conventional dermal patches include a carrier that holds a drug and allows the drug to be released onto a patient's skin for absorption. Many different kinds of dermal patches are known, including matrix type patches, reservoir-type patches, multi-laminate drug-in-adhesive type patches, and monolithic drug-in-adhesive type patches, and many others. Such patches can be readily prepared using technology which is known in the art such as described in Remington's Pharmaceutical Sciences, 18^{th} or 19^{th} editions, published by the Mack Publishing Company of Easton, Pa.

Several patents disclose the topical administration of a local anesthetic, particularly lidocaine, so that its pharmacological effects are restricted to the intracutaneous regions of drug penetration, with little or no systemic absorption. In each of these patents, the therapeutically effective topical amount of lidocaine is in the range of about 10 mg to 50 mg for delivery to the intact skin over a span of about 12 to 36 hours at a rate in the range of about 0.05 to 1 mg/cm² per hour, or about 0.5-40% by weight, based on the weight of the composition. For example,

U.S. Pat. No. 5,411,738 describes a method for the relief of pain of a host suffering from herpes zoster or post-herpetic neuralgia by inducing analgesia for an extended period of time, the method including maintaining lidocaine intradermally at a concentration sufficient to induce analgesia, at the site of the pain, whereby the pain is relived by lidocaine.

U.S. Pat. No. 5,776,952 describes a method for topical therapy of back pain, muscle tension and myofascial pain, which includes administering a topical carrier system containing a therapeutically effective amount of a local anesthetic, whereby the local anesthetic is applied to a region of skin lying beneath the topical carrier system.

U.S. Pat. No. 5,840,755 describes a method for topical therapy of headaches, which includes applying a therapeutically effective amount of a local anesthetic from a topical carrier system attached on forehead and/or temple skin.

U.S. Pat. No. 5,863,941 describes a periauricularly administered topical carrier system, which contains a therapeutic or preventative dose of a local anesthetic and a carrier substance for the treatment and prevention of pathological symptoms of the inner ear or labyrinth.

U.S. Pat. Nos. 6,455,066 and 6,746,689 describe a method for administering a local anesthetic including applying to a subject's skin a pharmaceutically acceptable topical formulation including a therapeutically effective amount of the local anesthetic, e.g., lidocaine, or a pharmaceutically acceptable salt thereof, and a penetration enhancing amount of an intradermal-penetration agent selected from the group consisting of a triglyceride (e.g., soybean oil), an aloe composition, and a mixture thereof, wherein the formulation is in a patch. The amount of local anesthetic in the topical formulation described is generally from about 1 to about 25 percent, preferably of from about 2 to about 20 percent, more preferably from about 3 to about 6 percent of the total weight of the formulation.

Endo Pharmaceuticals' Lidoderm® patch contains Lidocaine at a concentration of 5% by anesthetic volume. This patch is used to treat pain caused by shingles and other skin ailments. The anesthetic released by the Lidoderm® patch only penetrates into the skin deep enough to reach nerve endings proximate to the skin surface. Pain receptors located in deeper body tissue are not reached by the anesthetic and are untreated by the Lidoderm® patch.

U.S. Pat. Nos. 6,383,511 and 6,645,521, and published U.S. application No. 2004/0058994, each of which is incorporated herein by reference, disclose methods of treating pain receptors in the skin proximate to surgically closed wounds through topical delivery of a local anesthetic on or adjacent to an exterior surface of a surgically closed wound.

Many athletic injuries cause deep tissue damage and pain that cannot be treated with traditional topical anesthetics applied by patches. Additionally, if a subject has multiple injury locations, a patch must be applied to the patient over each site to treat the pain.

Therefore, there is a need for a patch for athletic injuries that can be applied locally, does not cause activity limiting and healing delaying side-effects, does not aggravate the injury site, is easy for a patient to apply, does not require professional administration or painful injections and allows a single administration to treat one or more injury sites for a prolonged period of time.

### BRIEF DESCRIPTION OF THE DRAWINGS

FIG. 1 is a top view of the contact surface of a treatment patch in accordance with the present invention; and

FIG. 2. is a sectional view taken along line 2-2 of FIG. 1.

### SUMMARY OF THE INVENTION

The invention provides a method for the topical treatment of pain induced by a sports injury, the method includes the step of applying to the skin of a subject in need thereof, a transdermal drug delivery system that includes a composition having a pain-relieving amount of a drug and a pharmaceutically acceptable carrier, wherein the transdermal drug delivery system is applied to skin at a site proximate to a sports injury site.

Furthermore, provided herein is a method for topical treatment of pain induced by a sports injury, the method including the step of applying to the skin of a subject in need thereof a transdermal drug delivery system that includes a composition having a pain-relieving amount of a drug and a pharmaceutically acceptable carrier, wherein the transdermal drug delivery system is applied to skin at a site regional to an sports injury site.

Additionally, provided herein is a sports injury and rehabilitative medicine analgesic patch including an overlay backing having an adhesive surface, wherein the adhesive surface of the backing includes an anesthetic area, and wherein the anesthetic area contains a composition including a pain-relieving amount of at least one local anesthetic in a pharmaceutically acceptable carrier.

Finally, the invention provides a sports injury and rehabilitative medicine analgesic dispenser system that includes an impermeable backing, a compartment, and an overlying permeable membrane, wherein the impermeable backing joins the permeable membrane at a circumferential edge of the dispenser, and wherein the compartment includes a composition having a pain-relieving amount of at least one local anesthetic in a pharmaceutically acceptable carrier.

### DETAILED DESCRIPTION OF THE INVENTION

The present invention is a sports injury and rehabilitation medicine analgesic patch to alleviate pain caused by sports-related injury. The concentrated local anesthetic in the patch penetrates deeply below the skin to the injury site to inhibit pain receptors throughout the damaged body tissue. The invention also relates to methods of treating sports injury pain using a topically applied patch.

The local anesthetic in the patch of the present invention is a suitable drug that provides local numbness or analgesia, or a drug that provides a regional blockage of nervous pathways that carry pain signals.

In another embodiment, two or more injuries can be treated by a single patch. In another embodiment, a combination of local anesthetics can be administered using a single patch.

In yet another embodiment, the compositions of the present invention can further include one or more additional compatible active ingredients which are aimed at providing the composition with another pharmaceutical effect in addition to that provided by a local anesthetic. “Compatible” as used herein means that the components of such a composition are capable of being combined with each other in a manner such that there is no interaction that would substantially reduce the efficacy of the composition under ordinary use conditions.

Such additional active ingredients include, but are not limited to penetration enhancers, and agents that reduce skin discomfort such as anti-inflammatory agents.

In one embodiment, the patch of the present invention is infused with penetration enhancers which aid in treatment effectiveness by facilitating delivery of the anesthetic. The term “penetration enhancer” as used herein refers to an agent known to accelerate the delivery of a substance through the skin. Suitable penetration enhancers usable in the present invention include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone^{RTM} from Whitby Research Incorporated, Richmond, Va.), alcohols, and the like. The permeation enhancer may also be a vegetable oil. Such oils include, for example, safflower oil, cottonseed oil and corn oil. Additional penetration enhancers may generally be found in Remington's Pharmaceutical Sciences, 18^{th} or 19^{th} editions, published by the Mack Publishing Company of Easton, Pa. which is incorporated herein by reference.

The patch of the present invention also can be infused with an anti-inflammatory agent to reduce skin discomfort. As used herein “inflammation” refers to a response to infection and injury in which cells involved in detoxification and repair are mobilized to the compromised site by inflammatory mediators. Thus, the body's response may include edema, vasodilation, fever and pain. The term “skin discomfort” is used herein to refer to burning, stinging, itching, tingling, loss of feeling or heightened sensitivity of the skin. “Steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Preferably, the additional active ingredients are added in a treatment-enhancing amount. As used herein a “treatment-enhancing amount” refers to an amount that is effective to accomplish the desired effect. Typically, such an effective amount is an amount between about 0.1 up to about 10 percent as weight per weight of the composition. More typically a treatment-enhancing amount would be between about 1 to about 5 percent.

Preferably, a treatment-enhancing amount of anti-inflammatory agent used to reduce skin discomfort is about 1 percent to about 5 percent, preferably about 1 percent to about 3 percent, and most preferably about 1 percent, wherein these percentages are expressed as weight per weight of the composition.

In other embodiment, the patch of the present invention can be used in conjunction with rehabilitation modalities, such as ultrasound and intophoresis. As used herein “intophoresis” refers to the introduction of a drug or additional active ingredient through intact skin by the application of a direct electric current

In an additional preferred embodiment, the patch of the present invention can be used in conjunction with rehabilitation therapies, such as heat, massage, manipulation, strength and stretching exercises, to maximize healing results with the elimination of muscle pain and spasm.

Other objects and features of the invention will become apparent as the description proceeds, especially when taken in conjunction with the accompanying drawings illustrating one embodiment of the invention.

The terms “treating” and “treatment” as used herein refer to reduction in severity and/or frequency of symptoms and/or their underlying cause, and improvement or remediation of damage.

The term “transdermal administration” as used herein refers to administration of a local anesthetic to the skin surface of a subject, including a human, so that the local anesthetic passes through the skin tissue and into the individual's blood stream, thereby providing a systemic effect.

The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably.

As used herein, the term “local anesthetic” means any drug that provides local numbness or analgesia or any drug that provides a regional blockage of nociceptive pathways (afferent and/or efferent). The local anesthetic can be any local anesthetic known or to be developed. Examples of local anesthetics suitable for use with the invention include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof. The amide and ester type local anesthetics are preferred. Amide type local anesthetics are characterized by an amide functionality, while ester type local anesthetics contain an ester functionality. Preferred amide type local anesthetics are: lidocaine, bupivacaine, prilocaine, mepivacaine, etidocaine, ropivacaine, dibucaine, and mixtures thereof. Preferred ester type local anesthetics are: tetracaine, procaine, benzocaine, chloroprocaine, their pharmaceutically acceptable salt, or a mixture thereof. The most preferred local anesthetic is lidocaine.

The meaning of “local anesthetic” as used herein also encompasses drugs not traditionally associated with local anesthetic properties but which have a local anesthetic effect, for example, non-narcotic analgesics, such as, acetylsalicylic acid, ketoprofen, piroxicam, diclofenac, indomethacin, ketorolac, rofecoxib, and celecoxib, and pharmaceutically acceptable salts thereof, or mixtures thereof.

The term “drug” means a substance used in the diagnosis, treatment, or prevention of a disease or medical condition or an active component of a medication. Of course, the term “drug” encompasses local anesthetics.

The composition of the present invention is a pharmaceutical composition. As used herein, the term “pharmaceutical composition” refers to a composition that is employed to prevent, reduce in intensity, cure or otherwise treat a target condition or disease. The pharmaceutical composition of the invention includes a drug carrier.

The terms “drug carrier”, “carrier”, or “vehicle” as used herein refers to carrier materials suitable for transdermal drug administration. Carriers and vehicles useful herein include any such materials known in the art which are nontoxic and do not interact with other components. As used herein the term “a pharmaceutically acceptable carrier” refers to any substantially non-toxic carrier conventionally useable for transdermal administration of pharmaceuticals in which a drug will remain stable and bioavailable. In one embodiment of the present invention, the local-anesthetic of the composition of the present invention comprises a pharmaceutically acceptable carrier to contain and deliver the active component to the application site.

Preferably, carriers are sterile and pharmaceutically acceptable for topical application and delivery of a drug into or through a patient's skin. Preferred functional characteristics of carriers are low adhesive strength, breathability, and conformability to the application area.

Pharmaceutically acceptable carriers for use in the invention are standard in the art, for example, matrix-type carriers, reservoir-type carriers, multi-laminate-type carriers, and monolithic drug-in-adhesive type carriers, such as those disclosed in TRANSDERMAL AND TOPICAL DRUG DELIVERY SYSTEMS (Tapash K. Ghosh et al. eds., 1997); see also Kristine Knutson and Lynn K. Pershing, Topical Drugs, in 2 REMINGTON: THE SCIENCE AND PRACTICE OF PHARMACY 866-885 (Alfonso R. Gennaro ed., 1995), the disclosures of which is hereby incorporated herein by reference.

In a preferred embodiment, the carrier is a matrix-type drug carrier. Matrix-type drug carriers are well known in the art. Suitable matrix-type drug carriers include, but are not limited to, the adhesives discussed below, such as polyisobutylene-based adhesives, silicone-based adhesives, and acrylic-based adhesives.

In another embodiment, the carrier is a hydrogel. Hydrogels are a mixture of water and a gelling agent, such as a hydrophilic polymer. In general, hydrogels form a three-dimensional lattice of polymer chains that retains an aqueous solution in a flexible, stable shape. Preferred hydrogels contain gelling agents distributed substantially uniformly throughout the carrier liquid, which is typically aqueous and may contain an alcohol and/or an oil.

Preferred gelling agents include, but are not limited to, crosslinked acrylic acid polymers such as carboxypolyalkylenes; hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Suitable hydrogels are commercially available, for example, suitable hydrogels can be purchased from BASF (St. Paul, Minn.) or Noveon (Cleveland, Ohio).

The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches of the present invention preferably comprise: (1) a backing layer, having an adhesive thereon; (2) a local-anesthetic component for local delivery of a local anesthetic, preferably, a local anesthetic in a carrier, referred to herein as a “local anesthetic composition”; The local-anesthetic components are collectively referred to herein as the “active components”. These components are described in more detail below.

Patches of the present invention can be any shape or size or can be customized to fit irregularly shaped body parts associated with sports injury, e.g. joints, back, arms, etc. For example, patches of the invention can be rectangular, square, round or oval in shape to treat the pain caused by sports injuries.

Patches suitable for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

The matrix patch comprises a drug containing matrix, an adhesive backing film overlay, and preferably, a release liner. In some cases, it may be necessary to include a impermeable layer to minimize drug migration into the backing film (e.g., U.S. Pat. No. 4,336,243, incorporated herein by reference). The drug-containing matrix is held against the skin by the adhesive overlay. Examples of suitable matrix materials include, but are not limited to, lipophilic polymers, such as polyvinyl chloride, polydimethylsiloxane, and hydrophilic polymers like polyvinylpyrrolidone, polyvinyl alcohol, hydrogels based on gelatin, or polyvinylpyrrolidone/polyethylene oxide mixtures.

The reservoir type patch design is characterized by a backing film coated with an adhesive and a reservoir compartment comprising a drug formulation, preferably in the form of a solution or suspension, that is separated from the skin by a semipermeable membrane (e.g., U.S. Pat. No. 4,615,699, hereby incorporated herein by reference). The adhesive coated backing layer extends around the reservoir's boundaries to provide a concentric seal with the skin and hold the reservoir adjacent to the skin.

The monolithic/single drug-in-adhesive patch design is characterized by the inclusion of the drug formulation in the skin contacting adhesive layer, a backing film, and preferably, a release liner. The adhesive functions both to release the anesthetic and adhere the anesthetic matrix to the skin. The drug-in-adhesive system does not require an adhesive overlay and thus the patch size is minimized. Also, drug-in-adhesive type patches are thin and comfortable (e.g., U.S. Pat. No. 4,751,087, incorporated herein by reference).

The multi-laminate drug-in-adhesive patch design further incorporates an additional semi-permeable membrane between two distinct drug-in-adhesive layers or multiple drug-in-adhesive layers under a single backing film. See Peterson, T. A. and Dreyer, S. J. 21 PROCEED. INTERN. SYMP. CONTROL. REL. BIOACT. MATER. 477-478 (Nice, France 1994), hereby incorporated herein by reference).

The backing layer or backing serves as the upper surface of the patch and functions as the primary structural element and provides the patch with its flexibility. The material selected for the backing material should be selected so that it is substantially impermeable to the local anesthetic and any other materials present; the backing is preferably made of a sheet or film of a flexible elastomeric material. The backing supports the active layers by way of an adhesive and holds the active layers against the application site. The combination of backing and adhesive should be biocompatible, non-irritating to the skin, breathable and able to hold the patch firmly against the skin.

Backings for use in patches of the invention are preferably made of a flexible, biocompatible material that imitates the elastic properties of skin and conforms to the skin during movement. Preferred have a moisture-vapor transmission rate similar to human skin. This reduces the chance of an infection developing under the patch after it is applied to a patient's skin.

Preferably, the backing layer is derived from synthetic polymers like polyolefin oils polyester, polyethylene, polyvinylidine chloride, and polyurethane or from natural materials like cotton, wool, etc. Non-occlusive backings allow the area to breathe (i.e., promote water vapor transmission from the skin surface). In one preferred embodiment, the backing film is an occlusive polyolefin foil (Alevo, Dreieich, Germany). The polyolefin foil is preferably about 0.6 to about 1 mm thick. Other suitable backings are commercially available; for example, suitable backings can be purchased from 3M (St. Paul, Minn.) and Bertek (St. Albans, Vt.).

Permeable membranes are used with patches of the present invention to overlay the portion of the patch adjacent to the skin to permit delivery of the patch's active ingredients to the application site. Preferably, the permeable membrane comprises a breathable material that is agreeable to the surface of a surgically closed wound and permits local delivery of local anesthetic into the skin of the patient at the wound site. Permeable membranes permit controlled delivery of the active components of the patch.

Permeable membranes useful in the present invention include thin non-porous ethylene vinyl acetate films or thin micro-porous films of polyethylene and polypropylene. Preferably, the permeable membrane is an ethyl vinyl acetate copolymer membrane. Suitable permeable membranes are commercially available; for example, suitable permeable membranes can be purchased from 3M (St. Paul, Minn.).

Adhesives are used with patches of the present invention to adhere the active components to the backing and to adhere the backing to the patient's application site. Preferably, adhesives useful in the present invention can function under a wide range of conditions, such as, high and low humidity, bathing, sweating etc. Adhesives for use with patches of the present invention are well known in the art and selection is readily accomplished by an ordinary practitioner. Suitable adhesives include, but are not limited to, polyisobutylene-based adhesives, silicone-based adhesives, and acrylic-based adhesives. Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; and hydrogels (e.g., high molecular weight polyvinylpyrrolidone and oligomeric polyethylene oxide).

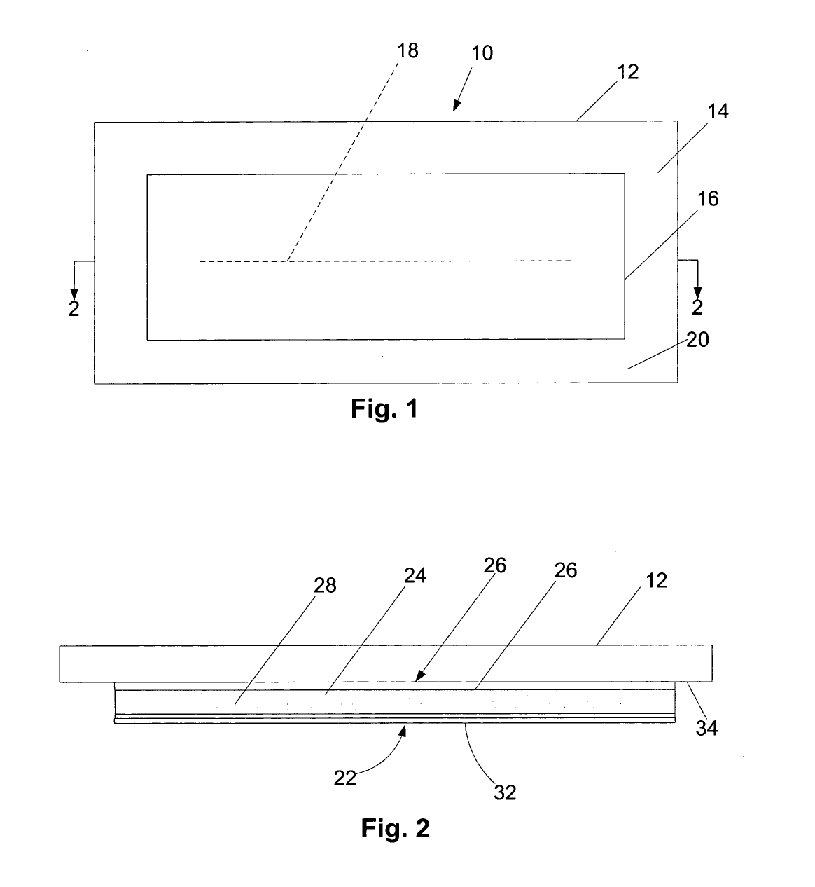

FIG. 1 shows the application surface of an sports injury treatment patch 10 of the present invention. Treatment patch 10 includes an elongate rectangular adhesive overlay backing 12 having an adhesive surface 14. Backing 12 is preferably comprised of a flexible, biocompatible material that imitates the elastic properties of skin and conforms to the skin during movement. Backing 12 is preferably breathable with a moisture vapor transmission rate similar to skin to reduce the chance of an infection developing under the patch after it is applied to a patient's skin. Adhesive surface 14 should be biocompatible, non-irritating to the skin, breathable and able to hold patch 10 firmly against the skin.

The adhesive surface 14 of backing 12 includes an elongate anesthetic area 16, centrally located on backing 12 and extending along the length of the backing. A local anesthetic of the type previously described is provided on area 16, typically in a carrier, for application over a sports injury site 18.

The anesthetic area 16 is surrounded by rectangular adhesive area 20 of backing 12. Area 20 is adhered to the skin of a patient adjacent injury site 18 to secure patch 10 in place over the injury site. The patch may be applied to a remote site.

When the patch is a adhered over injury site 18, local anesthetic from area 16 is applied directly to injury site 18 to treat pain receptors proximate to and through injury site 18.

FIG. 2 illustrates an elongate strip shaped first topical drug dispenser 22 of the present invention adhered to strip shaped area 16 of patch 10. First drug dispenser 22 typically contains a measured quantity of local anesthetic and a pharmaceutically acceptable drug carrier that is adhered to backing 12. The carrier may be a matrix type carrier, a reservoir type carrier, a multi-laminate carrier, a monolithic drug-in-adhesive or other type of carrier acceptable for topical application of a drug through a patient's skin.

In FIG. 2 first drug dispenser 22 is comprised of a matrix type drug carrier. Dispenser 22 is made up of a compartment 24 that contains a wall 26 that surrounds compartment 24. The compartment is filled with a hydrogel 28.

Methods to prepare formulations useful in the present invention are within the ordinary skill of persons skilled in the art.

In a preferred embodiment, hydrogel carrier 28 is a mixture of water and a hydrophilic polymer such as polyvinyl alcohol. Hydrogel 28 contains a three dimensional lattice of polymer chains that retains an aqueous solution in a flexibly stable shape. Hydrogel 28 is infused with an appropriately prepared aqueous solution containing a local anesthetic. Wall 26 includes an impermeable backing 30 and overlying permeable membrane 32. Impermeable backing 30 joins permeable membrane 32 at the circumferential edge of dispenser 22.

Permeable membrane 32 is preferably comprised of a breathable substance that is agreeable to the skin surface and permits local anesthetic to flow into injury site 18.

The preferred anesthetic solution infused into hydrogel 28 contains lidocaine at a concentration of about 4 percent to about 10 percent weight by weight of the composition for a hydrogel patch or about 10 percent to about 20 percent weight by weight of the composition for a matrix or a drug in adhesive patch.

If appropriate, a combination of local anesthetics can be infused into the hydrogel or other substances to improve the effectiveness of the patch.

If desired, patch 10 can be sterilized and packaged in a protective sheath (not shown). The sheath ensures patch sterility until opened. Sheet 34 is placed over backing 14 and dispenser 22 to prevent premature release of the anesthetic during patch storage and to keep the patch sanitary after the sheath is opened and before patch 10 is administered to a patient. Sheet 34 is comprised of a sanitary, impermeable flexible material and is easily removed from patch 10.

In use, patch 10 is preferably applied adjacent to the surface of an sports injury site 18 so that drug dispenser 22 is in proximity with sports injury site 18. Dispenser 22 overlies injury site 18 for effective administration of local anesthetic to sports injury site 18. Adhesive surface 14 is applied to a patient's skin adjacent injury site 18 to secure dispenser 22 in close contact with injury site 18.

Dispenser 22 releases a sustained dosage of a local anesthetic into injury site 18. Local anesthetic passes from hydrogel 28 though permeable membrane 32, through the skin and tissue and into the injury site 18. The local anesthetic penetrates into the injured body tissue to affect pain receptors proximate to the injured body tissue.

As used herein the term “pharmaceutically effective amount” “refers to the amount of any of the compositions of the present invention that result in a therapeutic or beneficial effect following its administration to a subject. The concentration of the substance is selected so as to exert its pharmaceutical effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound, composition or other active ingredient employed, the particular carrier utilized, and like factors.

As used herein the term “pain-relieving amount” refers to the amount of any of the compositions of the present invention that results in the reduction of pain in following its administration to a subject.

The appropriate dosages of local anesthetic for pain treatment by way of patches of the present invention is determined by a variety of factors. The rate at which the active components are absorbed is a function of skin permeability. Skin permeability varies between different sites on a patient's body and depends on the thickness of the stratum comeum. The stratum comeum is the outer-most layer of skin and is the main source of penetration and permeation resistance for dermally administered drugs. For example, the permeability, in general, increases in order from planter foot arch, lateral ankle, palm, ventral forearm, dorsal forearm, back, chest, thigh, abdomen, scalp, axilla, forehead, and scrotum; see R. C. Wester. & H. I. Maibach, Regional variation in Percutaneous Absorption, in PERCUTANEOUS ABSORPTION, MECHANISM, METHODOLOGY, DRUG DELIVERY 111-119 (R. L. Bronaugh & H. I. Maibach eds., 2nd ed. 1989), hereby expressly incorporated herein by reference. Anesthetic dose size and frequency of dosages should be determined by a trained medical professional and will depend on many factors, including patient weight, injury location, injury size and injury severity.

For treatment of surgically closed wounds, dose size and frequency of dosages should be determined by a trained medical professional and will depend on many factors, including patient weight, wound location, wound size, wound severity and the type of surgical closure.

The delivery rate of local anesthetic from a patch of the present invention that is required for proper pain relief is determined by a variety of factors. With reference to FIG. 2, one important factor regarding delivery rate is the surface areas of the local-anesthetic components in contact with a patient's skin. In general, the larger the contact surface area, the higher the rate of delivery. Different delivery rates of local anesthetic may be needed depending on the severity of pain caused by the wound. The surface areas of components can adjusted to provide the desired delivery rate of local anesthetic to a patient.

The dosage of local anesthetic administered by way of patches of the present invention can be controlled by the active surface area of the local-anesthetic component of the patch in contact with the skin. It is advantageous that several dosage strengths be available to the physician for his prescription, depending upon the severity of the pain. Thus, in general, a physician can adjust the local anesthetic dosage up or down by prescribing a patch having a local anesthetic component of larger or smaller surface area.

In general, the local anesthetic component of patches of the present invention will comprise a local anesthetic in an amount of from about 0.1 mg/cm² to about 50 mg/cm².

For hydrogel-type patches, the local anesthetic component of patches of the present invention will comprise a local anesthetic in an amount of about 0.5 mg/cm² to about 10 mg/cm², preferably, of about 2 mg/cm² to about 8 mg/cm², more preferably, of about 4 mg/cm² to about 6 mg/cm².

For matrix (drug-in-adhesive) type patches, preferably, the local anesthetic component of patches of the invention will comprise a local anesthetic in an amount of about 0.5 mg/cm² to about 30 mg/cm², preferably, of about 5 mg/cm² to about 25 mg/cm², more preferably, of about 8 mg/cm² to about 12 mg/cm².

For example, the rate of delivery of local anesthetic required-for proper pain relief is determined by the surface area of drug dispenser 22 in contact with a patient's skin. A drug dispenser having a larger surface area will dispense a larger dose of local anesthetic. Different dosages of local anesthetic may be needed depending on the severity of pain caused by the injury. The surface area of dispenser 22 is adjusted to provide the desired dose of local anesthetic to a patient.

Drug dispenser 22 is shaped to correspond to the skin proximate injured body tissue. This allows a precise application of the local anesthetic into the damaged body tissue to maximize the ability of the patch to deliver local anesthetic to pain receptors in the body tissue.

Patch 10 may be applied proximal to an injury site or regionally, i.e., near the site of the injury. For example the patch can be placed on the ankle for an ankle injury. It may be adhered to a patient through use of a drug-in-adhesive applied to the application surface of backing 12.

In one embodiment, disclosed patch 10 dispenses local anesthetic to the patient from drug dispensers 22. In some applications, the local anesthetic to be administered to the patient from the patch of the present invention may be secured to the patch backing without the necessity of a dispenser for the local anesthetic. For instance, an anesthetic contained in a paste or cream type topical drug formulation as disclosed in U.S. Pat. Nos. 6,383,511 and 6,645,521, which are incorporated herein by reference, may be spread on an adhesive backing 12 in area 16. The local anesthetic will flow directly through the skin and into the patient's circulatory system. The concentration of a local anesthetic contained in the drugs on the in a topical drug formulation would have to be properly adjusted.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

### Treatment of Sports Injury

Exemplary use of the present invention is described in a 63 year-old white male avid tennis player complaining of pain in the right shoulder and right lateral thigh with radiation. The pain was aggravated by repetitive sports activity e.g., tennis competition three times per week especially, “service motion”. Prior treatment history included cortisone injection into the pain “trigger zone” with temporary relief for 4 to 6 weeks. The pain caused sleep disturbance and, because of NSAID aggravated gastric irritation, the patient was prescribed Tylenol #3 at bedtime. Patient alleges the pain recurs between 2-3 AM, preventing the return to restful sleep. Examination revealed point tenderness in the right posterior rotator cuff and right lateral femoral tuberosity and facial tract. The diagnosis included right rotator cuff tendonitis and right lateral fasciaitis. The trigger points reproduced the painful symptoms.

Initial treatment with a new therapeutic approach utilizing a 5% lidocaine patch occurred in May 1997. Application of a 5% lidocaine patch at bedtime and during athletic activity resulted in dramatic improvement of symptoms with restoration of sleep routine. In 2001, repeat treatment of the patient with a 20% matrix based lidocaine patch resulted in increased penetration of the analgesic effect and improved regional pain relief. A five-year follow-up revealed continued symptomatic relief with the topical pain patch and continuation of athletic activity. Only one repeat injection of cortisone during the 5-year period was indicated.

### EXAMPLE 2

### Preparation of Prototypical Matrix Type 20% Lidocaine Patch

An adhesive matrix comprising a mixture of lidocaine in acrylate polymer adhesive is prepared in the following manner. About 60 g acrylate polymer (Durotak® 387-2052) and about 50 g lidocaine are dissolved in ethyl acetate. The concentration of lidocaine is about 15 wt/wt percent wet weight. The local-anesthetic component of the patch is then prepared by completely coating a 75 μm polyester film backing with the lidocaine acrylate matrix using a Warner Mathis thickness coater. The thickness of the wet film is about 270 μm. The coated film is dried at a rate of about three feet per minute through a 9 foot temperature zone at a temperature gradient of about 60° C. to about 90° C. in a KTF oven to evaporate the ethyl acetate yielding the local-anesthetic component. The dry adhesive lidocaine matrix film has a thickness of about 160 μm and the lidocaine concentration is about 20 wt/wt percent dry weight. The dry film is then laminated with a second sheet of 75 μm polyester film, thus giving the dry lidocaine matrix sandwiched between the two polyester films, referred to herein as the “lidocaine matrix laminate”. The lidocaine matrix laminate is run through a Mark Andy slitting and die cutting machine to size. These patches are sterilized and packaged using well-known methods.

While the analgesic patch of the present invention is directed to the treatment of athletic injuries, it also can be used to treat pain associated with other tissue injuries for which application of an systemic or local analgesic is insufficient. Systemic drugs have not shown much efficacy in relieving sports injury pain.

The present invention described hereinabove has both human and veterinary utility. The term “subject” as used herein includes animals of, avian, reptilian or mammalian origin. Preferably, subjects are mammals. Even more preferably, subjects are human.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. A method for topical treatment of pain induced by a sports injury, the method comprising the step: applying to skin of a subject in need thereof a transdermal drug delivery system comprising a composition comprising a pain-relieving amount of a drug and a pharmaceutically acceptable carrier, wherein the transdermal drug delivery system is applied to skin at a site proximate to a sports injury site.

2. The method according to claim 1, wherein the drug is at least one local anesthetic.

3. The method according to claim 2, wherein the at least one local anesthetic is selected from the group consisting of ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, acetylsalicylic acid, ketoprofen, piroxicam, diclofenac, indomethacin, ketorolac, rofecoxib, and celecoxib or a pharmaceutically acceptable salt thereof.

4. The method according to claim 3, wherein the local anesthetic is at least a pain-relieving amount of lidocaine.

5. The method according to claim 1, wherein the transdermal drug delivery system is a system selected from the group consisting of a monolithic drug-in adhesive system, a multilaminate drug-in adhesive system, a reservoir system and a matrix system.

6. The method according to claim 5, wherein the transdermal drug delivery system is a monolithic drug-in adhesive patch.

7. The method according to claim 6, wherein the pain relieving amount of the local anesthetic or a pharmaceutically acceptable salt thereof comprises about 0.5 mg/cm² to about 30 mg/cm².

8. The method according to claim 1, wherein the pharmaceutically acceptable carrier is a hydrogel.

9. The method according to claim 8, wherein the hydrogel comprises a cross-linked polymer selected from the group consisting of a crosslinked acrylic acid polymers, a hydrophilic polymer, a polyoxyethylene-polyoxypropylene copolymer and polyvinylalcohol; a cellulosic polymer; a gum; sodium alginate; and gelatin.

10. The method according to claim 8, wherein the pain-relieving amount of the local anesthetic or a pharmaceutically acceptable salt thereof comprises from about 0.5 mg/cm² to about 10 mg/cm².

11. The method according to claim 1, wherein the composition further comprises a treatment-enhancing amount of an additional active ingredient.

12. The method according to claim 11, wherein the additional active ingredient facilitates delivery of the at least one local anesthetic.

13. The method according to claim 12, wherein the additional active ingredient is a penetration enhancer.

14. The method according to claim 11, wherein the additional active ingredient reduces skin discomfort.

15. The method according to claim 14, wherein the additional active ingredient is a steroidal anti-inflammatory agent.

16. The method according to claim 1 wherein the method further comprises the step of administering a rehabilitation modality.

17. The method according to claim 16, wherein the rehabilitation modality is ultrasound.

18. The method according to claim 16, wherein the rehabilitation modality is intophoresis.

19. The method according to claim 1, wherein the method further comprises the step of administering a rehabilitation therapy.

20. The method according to claim 19, wherein the rehabilitation therapy is a therapy selected from the group consisting of a heat therapy, a massage therapy, a manipulation therapy, and an exercise therapy.

21. A method for topical treatment of pain induced by an sports injury, the method comprising the step applying to skin of a subject in need thereof a transdermal drug delivery system comprising a composition comprising a pain-relieving amount of a drug and a pharmaceutically acceptable carrier, wherein the transdermal drug delivery system is applied to skin at a site regional to an sports injury site.

22. The method according to claim 21, wherein the drug is a local anesthetic.

23. The method according to claim 22, wherein the local anesthetic is at least one local anesthetic selected from the group consisting of ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, acetylsalicylic acid, ketoprofen, piroxicam, diclofenac, indomethacin, ketorolac, rofecoxib, and celecoxib or a pharmaceutically acceptable salt thereof.

24. The method according to claim 23, wherein the at least one local anesthetic is at least a pain-relieving amount of lidocaine.

25. The method according to claim 21, wherein the transdermal drug delivery system is a system selected from the group consisting of a monolithic drug-in adhesive system, a multilaminate drug in adhesive system, a reservoir system and a matrix system.

26. The method according to claim 25, wherein the transdermal drug delivery system is a single layer drug-in adhesive patch.

27. The method according to claim 26, wherein the pain relieving amount of the local anesthetic or a pharmaceutically acceptable salt thereof comprises about 0.5 mg/cm² to about 30 mg/cm².

28. The method according to claim 21, wherein the pharmaceutically acceptable carrier is a hydrogel.

29. The method according to claim 28, wherein the hydrogel comprises a cross-linked polymer selected from the group consisting of a crosslinked acrylic acid polymers, a hydrophilic polymer, a polyoxyethylene-polyoxypropylene copolymer and polyvinylalcohol; a cellulosic polymer; a gum; sodium alginate; and gelatin.

30. The method according to claim 28, wherein the pain-relieving amount of the local anesthetic or a pharmaceutically acceptable salt thereof comprises from about 0.5 mg/cm² to about 10 mg/cm².

31. The method according to claim 21, wherein the composition further comprises a treatment-enhancing amount of an additional active ingredient.

32. The method according to claim 31, wherein the additional active ingredient facilitates delivery of the at least one local anesthetic.

33. The method according to claim 32, wherein the additional active ingredient is a penetration enhancer.

34. The method according to claim 31, wherein the additional active ingredient reduces skin discomfort.

35. The method according to claim 34, wherein the additional active ingredient is a steroidal anti-inflammatory agent.

36. The method according to claim 21, wherein the method further comprises the step of administering a rehabilitation modality.

37. The method according to claim 36, wherein the rehabilitation modality is ultrasound.

38. The method according to claim 36, wherein the rehabilitation modality is intophoresis.

39. The method according to claim 21, wherein the method further comprises the step of administering a rehabilitation therapy.

40. The method according to claim 39, wherein the rehabilitation therapy is at least one therapy selected from the group consisting of a heat therapy, a massage therapy, a manipulation therapy, and an exercise therapy.

41. An sports injury and rehabilitative medicine analgesic patch comprising an overlay backing having an adhesive surface, wherein the adhesive surface of the backing comprises an anesthetic area, and wherein the anesthetic area contains a composition comprising a pain-relieving amount of at least one local anesthetic in a pharmaceutically acceptable carrier.

42. The sports injury and rehabilitative medicine analgesic patch according to claim 41, wherein the at least one local anesthetic is at least one local anesthetic selected from the group consisting of ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, acetylsalicylic acid, ketoprofen, piroxicam, diclofenac, indomethacin, ketorolac, rofecoxib, and celecoxib or a pharmaceutically acceptable salt thereof.

43. The sports injury and rehabilitative medicine analgesic patch according to claim 42, wherein the local anesthetic is at least a pain-relieving amount of lidocaine.

44. The sports injury and rehabilitative medicine analgesic patch according to claim 41, wherein the pharmaceutically acceptable carrier is a hydrogel.

45. The sports injury and rehabilitative medicine analgesic patch according to claim 44, wherein the hydrogel comprises a cross-linked polymer selected from the group consisting of a crosslinked acrylic acid polymers, a hydrophilic polymer, a polyoxyethylene-polyoxypropylene copolymer and polyvinylalcohol; a cellulosic polymer; a gum; sodium alginate; and gelatin.

46. The sports injury and rehabilitative medicine analgesic patch according to claim 44, wherein the pain-relieving amount of the local anesthetic or a pharmaceutically acceptable salt thereof comprises from about 0.5 mg/cm² to about 10 mg/cm².

47. The sports injury and rehabilitative medicine analgesic patch according to claim 41, wherein the transdermal drug delivery system is a single layer drug-in adhesive patch.

48. The sports injury and rehabilitative medicine analgesic patch according to claim 47, wherein the pain relieving amount of the local anesthetic or a pharmaceutically acceptable salt thereof comprises about 0.5 mg/cm² to about 30 mg/cm².

49. The sports injury and rehabilitative medicine analgesic patch according to claim 41, wherein the composition further comprises a treatment-enhancing amount of an additional active ingredient.

50. The sports injury and rehabilitative medicine analgesic patch according to claim 49, wherein the additional active ingredient facilitates delivery of the at least one local anesthetic.

51. The sports injury and rehabilitative medicine analgesic patch according to claim 50, wherein the additional active ingredient is a penetration enhancer.

52. The sports injury and rehabilitative medicine analgesic patch according to claim 49, wherein the additional active ingredient reduces skin discomfort.

53. The sports injury and rehabilitative medicine analgesic patch according to claim 52, wherein the additional active ingredient is a steroidal anti-inflammatory agent.

54. A sports injury and rehabilitative medicine analgesic dispenser system comprising an impermeable backing, a compartment, and an overlying permeable membrane, wherein the impermeable backing joins the permeable membrane at a circumferential edge of the dispenser, and wherein the compartment comprises a composition comprising a pain-relieving amount of at least one local anesthetic in a pharmaceutically acceptable carrier.

55. The sports injury and rehabilitative medicine analgesic dispenser system according to claim 54 wherein the analgesic dispenser system is a monolithic drug in adhesive patch.

56. The sports injury and rehabilitative medicine analgesic dispenser system according to claim 55, wherein the pain-relieving amount of the local anesthetic or a pharmaceutically acceptable salt thereof comprises from about 0.5 mg/cm² to about 30 mg/cm².

57. The sports injury and rehabilitative medicine analgesic dispenser system according to claim 54, wherein the at least one local anesthetic is selected from the group consisting of ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, acetylsalicylic acid, ketoprofen, piroxicam, diclofenac, indomethacin, ketorolac, rofecoxib, and celecoxib or a pharmaceutically acceptable salt thereof.

58. The sports injury and rehabilitative medicine analgesic dispenser system according to claim 57, wherein the local anesthetic is at least a pain-relieving amount of lidocaine.

59. The sports injury and rehabilitative medicine analgesic dispenser system according to claim 54, wherein the pharmaceutically acceptable carrier is a hydrogel.

60. The sports injury and rehabilitative medicine analgesic dispenser system according to claim 59, wherein the hydrogel comprises a cross-linked polymer selected from the group consisting of a crosslinked acrylic acid polymers, a hydrophilic polymer, a polyoxyethylene-polyoxypropylene copolymer and polyvinylalcohol; a cellulosic polymer; a gum; sodium alginate; and gelatin.

61. The sports injury and rehabilitative medicine analgesic dispenser system according to claim 59, wherein the pain-relieving amount of the local anesthetic or a pharmaceutically acceptable salt thereof comprises from about 0.5 mg/cm² to about 10 mg/cm².

62. The sports injury and rehabilitative medicine analgesic dispenser system according to claim 54, wherein the composition further comprises a treatment-enhancing amount of an additional active ingredient.

63. The sports injury and rehabilitative medicine analgesic dispenser system according to claim 62, wherein the additional active ingredient facilitates delivery of the at least one local anesthetic.

64. The sports injury and rehabilitative medicine analgesic dispenser system according to claim 63, wherein the additional active ingredient is a penetration enhancer.

65. The sports injury and rehabilitative medicine analgesic dispenser system according to claim 62, wherein the additional active ingredient reduces skin discomfort.

66. The sports injury and rehabilitative medicine analgesic dispenser system for according to claim 65, wherein the additional active ingredient is a steroidal anti-inflammatory agent.
